(19) United States
(12) Patent Application Publication
(10) Pub. No.: US 2013/0203617 A1
(43) Pub. Date: Aug. 8, 2013

(54) Glycoprotein Analysis Kit and Use Thereof

(71) Applicants: Hak Sung Kim, Daejeon (KR); Sun Young Park, Daejeon (KR); Sang Chul Lee, Daejeon (KR); Eun Kyoung Kim, Daejeon (KR); Sung Ho Kim, Daejeon (KR); Wan Jung Kim, Daejeon (KR); Yong Tae Kim, Daejeon (KR); Young Dok Son, Daejeon (KR); Jung Hoe Kim, Daejeon (KR)

(72) Inventors: Hak Sung Kim, Daejeon (KR); Sun Young Park, Daejeon (KR); Sang Chul Lee, Daejeon (KR); Eun Kyoung Kim, Daejeon (KR); Sung Ho Kim, Daejeon (KR); Wan Jung Kim, Daejeon (KR); Yong Tae Kim, Daejeon (KR); Young Dok Son, Daejeon (KR); Jung Hoe Kim, Daejeon (KR)

(73) Assignee: KOREA ADVANCED INSTITUTE OF SCIENCE AND TECHNOLOGY, Daejeon (KR)

(21) Appl. No.: 13/695,197

(22) PCT Filed: Apr. 29, 2011
(86) PCT No.: PCT/KR2011/003214

§ 371 Date: Jan. 30, 2013

(30) Foreign Application Priority Data
Apr. 29, 2010 (KR) 10-2010-0040185

(51) Int. Cl. G01N 33/74; C12N 15/85; G01N 33/68

(52) U.S. Cl. CPC G01N 33/746; G01N 33/6842; C12N 15/85
USPC 506/9; 436/501; 435/7.21; 435/360

(57) ABSTRACT

Disclosed are a glycoprotein analysis kit and use thereof. The glycoprotein analysis kit comprises a fluorescently labeled antibody, a fluorescently labeled biomaterial, and a support, and is used in a dual probing method for analyzing the content of a glycoprotein and profiling the oligosaccharide chain, simultaneously, and a method for selecting a single cell producing the glycoprotein having a desired glycosylation pattern. Also, a single cell producing the glycoprotein having a desired glycosylation pattern is provided.

### TECHNICAL FIELD

The present invention relates to a glycoprotein analysis kit and use thereof. More particularly, the present invention relates to a glycoprotein analysis kit comprising a fluorescently labeled antibody, a fluorescently labeled biomaterial, and a support, a dual probing method for analyzing the content of a glycoprotein and profiling the oligosaccharide chain simultaneously with using the glycoprotein analysis kit, a method for selecting a single cell producing the glycoprotein having a desired glycosylation pattern using the glycoprotein analysis kit, and a single cell producing the glycoprotein having a desired glycosylation pattern.

### BACKGROUND

Glycoproteins are a generic term for proteins that contain oligosaccharide chains (carbohydrates) covalently attached to polypeptide side-chains. They account for most of the protein-based therapeutics that have been approved thus far or are candidates under development. Profiles of the oligosaccharide chains, that is, the kinds of linking structures of monosaccharides constituting the oligosaccharide chains, are different from one cell to another, from one tissue to another, and from one species to another. The profile of the oligosaccharide chains is known to act as an important factor to determine the various biological functions of the glycoprotein including biological activity, immune response, pharmacokinetics, etc. Representative among the glycoproteins, the functions of which are affected greatly by oligosaccharide chains are antibodies, interferon, hormones, and erythropoietin (hereinafter referred to as “EPO”). Particularly, EPO is known to have longer serum half-life and thus greater in vivo activity with a higher content of terminal sialic acid moiety. In practice, novel forms of EPO having an increased sialic acid content have been developed and used as therapeutics.

Typically, therapeutic glycoproteins are produced by introducing a relevant gene to an animal cell capable of oligosaccharide chains similar to those of human, and repetitive process of selecting clones monoclonal cell lines producing glycoproteins with the desired oligosaccharide chains. Enzyme-linked immunosorbent assay (ELISA) in conjugation with Limiting dilution cloning (LDC) is one of the most frequently used methods for selecting monoclonal cell lines. In this method, a cell suspension is diluted and plated at a cell density of homogeneous cell per well into 96-well microplates and cultured, after which the supernatant of culture medium is subjected to ELISA to quantify protein levels. Although it is widely used due to simplicity, this method suffers from the disadvantage of being labor intensive. Thus, many studies have been done so as to overcome the disadvantage.

Gel microdrop technology was suggested in which agarose microdrops encapsulate individual cells and use fluorescently labeled antibodies to detect the protein produced by the cells (Nature 1997, 3, 583). However, encapsulation of single cells within microdrops is less likely to occur and requires special techniques and instruments. In an alternative method, cells are encapsulated with biotin, which is then used to capture biotin-labeled antibodies using avidin as a bridge (J. Immunol. Methods 1999, 230, 141). Due to comprising a cell modification process, however, this method is impossible to apply to cells susceptible to cell modification, and it is required to set forth optimal conditions for each cell line.

Recently developed was a Poly(dimethylsiloxane) (PDMS) microwell plate which is designed to segregate individual cells within microwells, each having micrometer diameter and depth, so that proteins produced from the single cell can be analyzed. As an application, a single cell-based microwell array was reported to isolate leukocytes selectively binding to a certain antigen (Cytometry A 2007, 71A, 1003). Further, the application of a microfluidics device to microwells allowed the selection of cells reactive or affinitive to certain materials (Lab Chip 2005, 5, 1380). Similarly, a microengraving method was suggested in which cells are grown within microwells and proteins produced within each microwell are transferred in the form of a microarray onto a glass so that single cells that produce an antibody recognizing a specific antigen of interest are selectively isolated to establish a new cell line (Nat. Biotechnol. 2006, 24, 703). These methods using microwells are applicable to a broad spectrum of cell lines and can analyze a large number of cells in one practice, thus selecting only cells of interest within a short time. However, because conventional methods cannot conduct oligosaccharide chain profiling in cells producing glycoproteins, the oligosaccharide chain profiling require additional processes for analyzing monosaccharide content and chain structures for their selection. To date, the oligosaccharide chain profiling is a complex and time- and labor-intensive process. Particularly, sialic acid is very difficult to quantitatively analyze due to its high negative charge (Biochim. Biophys. Acta 2006, 1764, 1853), thus acting as a large barrier to the development of therapeutic glycoproteins.

### DISCLOSURE

### Technical Problem

Leading to the present invention, intensive and thorough research into an analysis method for determining the quantity of a glycoprotein produced by a single cell and the content of a specific carbohydrate in the glycoprotein without requiring additional complex oligosaccharide chain profiling processes, conducted by the present inventors, resulted in the finding that a glycoprotein analysis kit comprising a fluorescently labeled antibody selectively binding to a protein moiety of a glycoprotein of interest, a fluorescently labeled biomaterial selectively binding to a carbohydrate moiety of the glycoprotein of interest, and a support for immobilizing the glycoprotein of interest thereto allowed the simultaneous analysis of the quantity of the glycoprotein produced by the single cell and the content of a specific carbohydrate in the glycoprotein, the inventors of the present invention further found that fluorescence intensities directing to the level of the glycoprotein and the content of the specific carbohydrate in the glycoprotein are determined by a dual probing method and are statistically analyzed to predict the level of the glycoprotein and the content of the specific carbohydrate, so that single cells capable of producing glycoproteins having a desired glycosylation pattern can be rapidly selected and isolated to establish new cell lines.

### Technical Solution

It is object of the present invention to provide a glycoprotein analysis kit comprising fluorescently labeled antibody, a fluorescently labeled biomaterial, and a support.

It is another object of the present invention to provide a dual probing method for quantifying a glycoprotein and profiling glycosylation simultaneously, using said glycoprotein analysis kit.

It is a further object of the present invention to provide a method for selecting a single cell producing a glycoprotein having a desired glycosylation pattern, using the glycoprotein analysis kit and the dual probing method.

It is still a further object of the present invention to provide a cell producing a glycoprotein having a desired glycosylation pattern, selected by the dual probing method therein.

### Advantageous Effects

As described above, the fluorescent signals detected from the fluorescently labeled antibody selectively binding to a protein moiety of a glycoprotein of interest and the fluorescently labeled biomaterial selectively binding to a carbohydrate moiety of the glycoprotein of interest, both contained in the glycoprotein analysis kit of the present invention allowed the simultaneous analysis of the quantity of the glycoprotein produced by the single cell and the content of a specific carbohydrate in the glycoprotein without requiring additional analysis processes. When used in conjugation with various kinds of biomaterials (including lectins, aptamers, peptides, antibodies etc.), the kit can analyze contents of various carbohydrates. In addition, fluorescence intensities detected by the glycoprotein analysis kit of the present invention are statistically analyzed to predict the level of the glycoprotein and the content of the specific carbohydrate and select single cells capable of producing glycoproteins having a desired glycosylation pattern. The single cells can be isolated from a group of cell using a micropipette and used to establish new cell lines. Therefore, the glycoprotein analysis kit of the present invention can be used to select cells capable of producing therapeutic glycoproteins with desired glycosylation patterns and are industrially applicable for establishing cell lines producing therapeutic glycoprotein.

### BRIEF DESCRIPTION OF THE DRAWINGS

The above and other objects, features and other advantages of the present invention will be more clearly understood from the following detailed description taken in conjunction with the accompanying drawings, in which:

FIG. 1 is a schematic view illustrating a method for the quantitative analysis of a glycoprotein in accordance with the present invention;

FIG. 2 is a schematic view illustrating a method for the selection of a single cell capable of producing a glycoprotein with a desired glycosylation pattern;

FIG. 3 shows graphs and photographs of fluorescent signal intensities as quantitatively analyzed by the glycoprotein analysis kit of the present invention;

FIG. 4 shows photographs of fluorescent signals as measured by the glycoprotein analysis kit for selecting single cells in accordance with the present invention; and

FIG. 5 shows graphs of distributions of fluorescence intensities as obtained by a statistical analysis method for the selection of single cells.

### BEST MODE

In accordance with an aspect thereof, the present invention addresses a glycoprotein analysis kit comprising (A) a fluorescently labeled antibody selectively binding to a protein moiety of a glycoprotein of interest; (B) a fluorescently labeled biomaterial selectively binding to a carbohydrate moiety of the glycoprotein of interest; and (C) a support for immobilizing the glycoprotein of interest thereto.

As used herein, the term “a protein of interest” is intended to refer to a target protein which is analyzed for glycosylation pattern and protein levels, and may include, but is not limited to, purified glycoproteins or glycoproteins isolated from single cells. Preferable are antibodies, interferon, hormones and Erythropoietin (EPO).

As used herein, the term “antibody” refers to a protein that is produced by the immune system in response to an antigen stimulus and specifically binds to a specific antigen during vigilance movement through blood and lymph to exert an antigen-antibody reaction. If the binding of the antibody used in the kit of the present invention to an epitope interferes with the binding between the biomaterial and the carbohydrate moiety of the glycoprotein, the quantitative analysis of the carbohydrate content of the glycoprotein cannot be performed with precision. Thus, the antibody is preferably selected so as to recognize an epitope having no influences on the binding of the biomaterial. Examples of the antibody include polyclonal antibodies, monoclonal antibodies, recombinant antibodies, multivalent antibodies, and multispecific antibodies. Antibody production is well known to those skilled in the art. Polyclonal antibodies may be produced by injecting an antigen to an animal and purifying them from the serum of the animal. Polyclonal antibodies are multiple antibodies that bind to the same antigen, and can be produced from any animal host such as goats, rabbits, sheep, monkeys, horses, pigs, cows, dogs, etc. but not limited to. Monoclonal antibodies are antibodies obtained from homogenous groups. Monoclonal antibodies are monovalent for the same antigenic site, that is, the same epitope, and thus show very high specificity for their antigens. They can be produced using a well-known technique, such as the hybridoma method (Kohler and Milstein (1976) European Journal of Immunology 6:511-519), or the phage antibody library method (Clackson et al, Nature, 352:624-628, 1991; Marks et al, J. Mol. Biol., 222:58, 1-597, 1991). In addition, the antibody in the present invention may be a complete antibody composed of two full-length light chains and two full-length heavy chains or a functional fragment. This functional fragment means a fragment retaining an antigen binding function, and includes Fab, F(ab′), F(ab′)2, and Fv.

As used herein, the term “biomaterial” means a material that binds to a saccharide moiety. So long as it binds to the saccharide moiety of the glycoprotein of interest, any biomaterial may be used in the present invention. Examples include lectins, antibodies, aptamers or peptides that recognize specific carbohydrate structures. Lectins are carbohydrate-binding proteins that are highly specific for their sugar moieties and serve to quantify certain carbohydrates. Lectins occur ubiquitously in nature, and are found in vertebrates, microbes, and viruses, but predominantly in plants. Available among the lectins of plant origin are concanavalin A (ConA) specific for mannose, Maackia Amurensis agglutinin (MAA) for sialic acid, Ricinus communis agglutinin (RCA) for N-acetylgalactosamine (GalNAc), Aleuria aurantia lectin (AAL) for L-fucose, and wheat germ agglutinin (WGA) for N,N-diacetyl chitobios (di-GlcNAc). Preferred is MMA binding to the terminal sialic acid of a saccharide chain. Sialic acid is known to play an important role in the function and stability of most glycoproteins. Thus, the content of sialic acids in glycoproteins can correlate to the stability of the glycoproteins of which allow protein to be isolated.

The term “fluorescently labeled,” as used herein, is intended to refer to a condition in which the antibody or the biomaterial becomes fluorescent as a fluorescent label is bound thereto. Examples of the fluorescent label useful in the present invention include, but are not limited to, rhodamines such as rhodamine, TAMRA, etc.; fluoresceins such as fluorescein, fluorescein isothiocyanate (FITC) and fluorescein amidite (FAM); boron-dipyrromethene (bodipy); alexa fluor; and cyanine dyes such as Cy3, Cy5, Cy7, and indocyanine green, with preference for cyanine dyes having an NHS-ester terminal group reactive selectively to an amine group. Given the condition that the antibody and the biomaterial are fluorescently labeled with respective fluorescent dyes which are different from each other in absorption and emission wavelengths, the fluorescent signals generated from them are independently detected to analyze the antibody and the biomaterial simultaneously in the present invention.

As used herein, the term “support” means a medium for immobilizing the glycoprotein of interest thereto. A non-limiting, illustrative example includes a glass substrate. Preferable is a glass substrate coated with nitrocellulose or nylon to allow the glycoprotein of interest to be bound thereto or with an antibody capable of binding to the glycoprotein of interest. Preferably, this antibody recognizes an epitope other than that to which the fluorescently labeled antibody binds.

In accordance with another aspect thereof, the present invention addresses a method for quantitating a glycoprotein of interest, comprising (A) reacting a glycoprotein of interest immobilized onto a support sequentially with a fluorescently labeled antibody binding selectively to a protein moiety of the glycoprotein of interest and a fluorescently labeled biomaterial binding selectively to a carbohydrate moiety of the glycoprotein of interest, sequentially; and (B) measuring fluorescent signals generated from the antibody and the biomaterial, both being associated with the support.

As used herein, the term “fluorescent signal” means the intensity of fluorescence generated from the fluorescently labeled antibody and biomaterial bound to the glycoprotein captured by the support.

After reacting with the fluorescently labeled antibody and the fluorescently labeled biomaterial, the method of the present invention may further comprise washing the support to remove the antibody and the biomaterial which remain unreacted, so as to detect only the fluorescent signal from the captured glycoprotein.

In addition, the method of the present invention may further comprise analyzing the measured fluorescent signal to determine the level of the glycoprotein and profiling the glycosylation of the glycoprotein simultaneously.

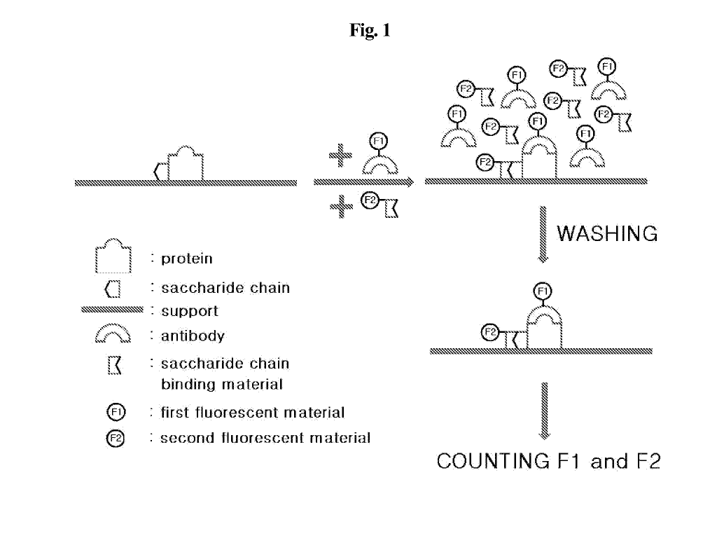

This dual probing method for the quantitative analysis of a glycoprotein according to the present invention will be described in detail with reference to FIG. 1. FIG. 1 is a schematic view of the dual probing method for quantitatively analyzing a glycoprotein.

First, a glycoprotein of interest is purified and isolated, and then immobilized onto a support such as a glass substrate. In this context, the support may be coated with nitrocellulose or nylon or with an antibody recognizing the glycoprotein to facilitate the support's capturing the glycoprotein. Next, an antigen-antibody reaction is induced by adding a fluorescently labeled antibody specific for the glycoprotein immobilized onto the support. After completion of the antigen-antibody reaction, the support is washed to remove the fluorescently labeled antibody which remains unreacted. Subsequently, a biomaterial that is capable of binding to a carbohydrate moiety of the glycoprotein and is labeled with a different fluorescent material is reacted with the glycoprotein. Binding affinity is typically at the level of nanomoles (˜nM) between antigen and antibody whereas being at the level of micromoles (˜μM) between the carbohydrate moiety and the biomaterial. Because of this relatively weak binding affinity, the amount and reaction time of the biomaterial should be optimized to guarantee a sufficient reaction of the hydrocarbonate moiety with the biomaterial. After the reaction is completed, the fluorescently labeled biomaterial that remained unreacted is removed by washing the support. Thereafter, fluorescent signals from both the fluorescently labeled antibody and the fluorescently labeled biomaterial are independently measured, and the quantitative analysis of the glycoprotein can be implemented on the basis of the fluorescent signals. In this context, the glycoprotein of interest may be analyzed for protein level and glycosylation pattern, e.g., the number of carbohydrate per molecule of the glycoprotein.

In one embodiment of the present invention, recombinant human EPO with various sialic acid contents were analyzed at various concentrations using a fluorescently labeled antibody specific for the EPO and a fluorescently labeled MAA lectin specific for sialic acid. The fluorescent intensity of the antibody bound to the rhEPO was proportional to the concentration of the rhEPO. For example, the fluorescent intensity increased with the increase of EPO level. Thus, the fluorescently labeled antibody is effective for quantitating EPO. In detail, given that a standard curve of the fluorescent intensity of the antibody is constructed against already known concentrations of a glycoprotein, a fluorescent signal from an unknown concentration of the glycoprotein can be applied to the standard curve to determine the concentration. Separately, the fluorescent signal of MAA was increased with an increase in the sialic acid content when the concentration of EPO was constant, and with an increase in the concentration of the protein when the sialic acid content was constant. This result indicates that the fluorescent intensity of lectin increases in proportion to the total sialic acid content of the entire glycoprotein. Because the fluorescent signal of the lectin is dependent on the content of a specific carbohydrate in the glycoprotein, the number of the specific carbohydrate per molecule of the glycoprotein can be determined if the concentration of the glycoprotein is known. That is, given that a standard curve of the fluorescent intensity of the biomaterial is constructed against already known numbers of a specific carbohydrate per molecule of the glycoprotein, an unknown number of the specific carbohydrate can be determined by comparing the fluorescent signal measured from the specific carbohydrate against the standard curve.

In accordance with a further aspect thereof, the present invention is directed to a method for the selection of a single cell producing a glycoprotein with a desired glycosylation pattern, comprising (A) culturing cells individually, said cells including the single cell; (B) bringing a support coated with an antibody specific for the glycoprotein into contact with the cells to transfer the glycoprotein secreted from the cells onto the support; (C) reacting with the glycoprotein-captured support with a fluorescently labeled antibody selectively binding to a protein moiety of the glycoprotein and a fluorescently labeled biomaterial selectively binding to a carbohydrate moiety of the glycoprotein, sequentially; (D) statistically analyzing fluorescent signals level from the support antibody and the biomaterial; and (E) determining the single cell having a desired glycosylation pattern, based on the analysis of the fluorescent signals from the cells.

As used herein, the term “single cell or homogeneous cell” means a cell which is derived from one ancestor and has the same morphology and properties.

After the step of reacting the support with the fluorescently labeled antibody and the fluorescently labeled biomaterial sequentially, the method of the present invention may further comprise washing the support to remove the antibody and the biomaterial, both remaining unreacted, to detect exact fluorescent signals, and measuring fluorescent signals from the fluorescently labeled antibody and the fluorescently labeled biomaterial, both being bound to the support.

Optionally, the method of the present invention may further comprise establishing a cell line producing the glycoprotein having a desired glycosylation pattern through continual passage, after the step of determining the single cell.

The statistical analysis of fluorescent signals may be carried out preferably by, but is not limited to, (i) processing measurements of fluorescent signals to set forth a normal distribution for a population distribution; and (ii) subjecting a level of the glycoprotein produced from each single cell and a number of a specific carbohydrate per molecule of the glycoprotein to relative comparison.

Most of the pharmaceutically active proteins that have been studied or are being studied are accounted for by glycoproteins. Typically, with the higher content of carbohydrate moiety therein, a glycoprotein is more stable and thus retains the pharmaceutical activity for a longer period of time. Accordingly, the production of a glycoprotein rich in oligosaccharide may be a strategy for improving the pharmaceutical activity of the glycoprotein. In addition, since the pharmaceutical activity of a glycoprotein may vary depending on its carbohydrate content, it may be important to determine a carbohydrate content optimal for the pharmaceutical activity of the glycoprotein.

Also, the carbohydrate content of a glycoprotein is greatly affected by the cell expressing the glycoprotein. Therefore, it is advantageous in terms of pharmaceutical activity to select cells producing a glycoprotein with a high or optimal carbohydrate content and to produce the glycoprotein from the cells.

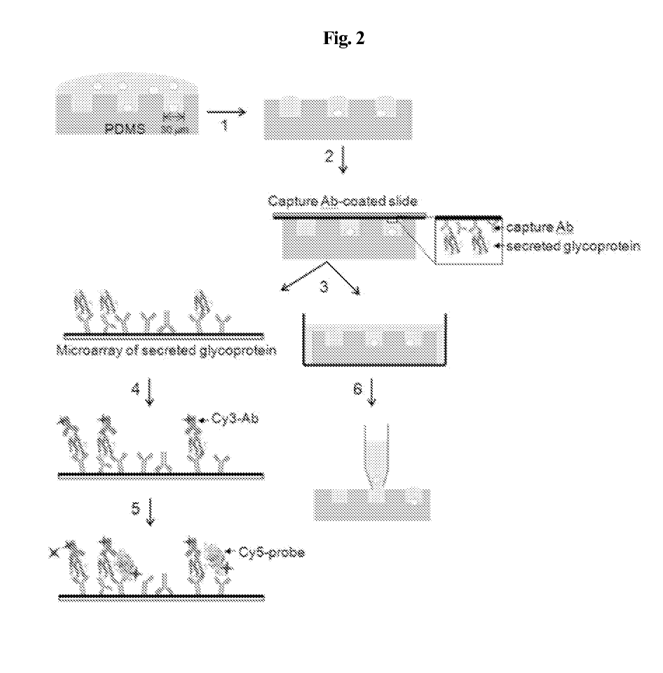

With reference to FIG. 2, a method for the selection of a single cell producing a glycoprotein having a desired glycosylation pattern will be described in detail. FIG. 2 is a schematic view illustrating the selection of a single cell producing a glycoprotein having a desired glycosylation pattern.

Cells are distributed to PDMS microwell plates to assign one cell per microwell and cultured. The microwell plates are overlaid with a glass substrate coated with a primary antibody specific for a glycoprotein and incubated at 37° C. for 1 hr, so that the glycoproteins secreted from the single cell within each microwell are transferred in a microarray pattern onto the glass substrate. Then, the glass substrate is reacted with a fluorescently labeled antibody specific for a protein moiety of the glycoprotein of interest and a fluorescently labeled biomaterial binding selectively to a carbohydrate moiety of the glycoprotein of interest, sequentially, after which respective fluorescent signals from the fluorescently labeled antibody and the fluorescently labeled biomaterial are measured. In this context, the measurements of the fluorescent signals from the antibody and the biomaterial in each well accounts respectively for a relative amount of the glycoprotein of interest produced by the single cell and a total carbohydrate content, that is, the product of a carbohydrate content per molecule of the glycoprotein of interest by the concentration of the glycoprotein in each well. Hence, the carbohydrate content per molecule of the glycoprotein is calculated by dividing the total carbohydrate content by the concentration of the glycoprotein, and thus is proportional to the fluorescent signal of the biomaterial divided by that of the antibody. To obtain a graph representing a statistically analyzable normal distribution, measurements of the fluorescent signals from the antibody and the biomaterial are converted into log scales. A graph is constructed for the glycoproteins produced by individual cells by plotting ratios of the log value of the fluorescent signal of the biomaterial to the long value of by the fluorescent signal of the antibody set forth on the Y-axis against log values of the fluorescent signal of the antibody on the X-axis. In this plot, the quantity of the glycoprotein of interest produced in each cell and the content of a specific carbohydrate per molecule of the glycoprotein can be analyzed. Based on the analysis result, single cells producing the glycoprotein of interest can be discriminated according to the carbohydrate content per glycoprotein unit while single cells producing a glycoprotein with a desired glycosylation pattern can be selected.

Optionally, the method of the selection of a single cell may further comprise (A) treating the selected single cell with trypsin; and (B) isolating the trypsinized single cell using a micropipette.

The term “micropipette,” as used herein, means a laboratory tool comprising a capillary tube fabricated in a micropipette puller. The micropipette is preferably designed to have a diameter of 50 μm in consideration of the diameter and inter-well distance of microwells. Since the diameter of the micropipette is dependent on the speed of the micropipette puller and temperature, the conditions must be optimized to set a desired diameter.

In the method, trypsin is used to weaken the adherence of single cells to the microwell while the single cells detached from the microwell are isolated using the capillary phenomenon of the micropipette. When trypsin is used at too high concentration, single cells are randomly isolated from the microwells, due to the capillary phenomena, only the selected single cells cannot be harvested. Thus, the concentration of trypsin must be so exact as to isolate single cells using capillary phenomenon.

In accordance with still a further aspect thereof, the present invention addresses a cell, isolated using the method of the selection of a single cell which produces a glycoprotein having a desired glycosylation pattern.

From many cells, single cells producing glycoproteins with desired glycosylation patterns can be effectively selected by profiling oligosaccharide chains of the glycoproteins. That is, in order to select single cells producing glycoproteins with high carbohydrate content, the ratio of the fluorescent intensity of lectin to the fluorescent intensity of the antibody must be taken into consideration. A single cell with the highest ratio may be used to establish a cell line which produces a glycoprotein with a high carbohydrate content.

In one embodiment, single cells producing a higher level of EPO with a higher content of sialic acid were established by repeating the selection method and the isolation method more times (Tables 1 and 2).

### MODE OF THE INVENTION

A better understanding of the present invention may be obtained through the following examples which are set forth to illustrate, but are not to be construed as limiting the present invention.

### Example 1

### Simultaneous Analysis of the Level of Glycoprotein and the Content of Specific Carbohydrate Using Glycoprotein Analysis Kit

### Example 1-1

### Preparation of Fluorescently Labeled Antibody

The polyclonal anti-EPO-antibody was labeled with the fluorescent dye Cy3-NHS ester to afford Cy3-α-EPO. In 500 μL of PBS, 100 μL of the polyclonal anti-EPO-antibody (sigma, 2 mg/mL) and one vial of Cy3-NHS ester (GE healthcare) were mixed. Of the resulting mixture, 200 μL was placed in an EP tube. After incubation at room temperature for 1 hr, the tube was centrifuged 3-4 times against a microfilter (Microcon YM-100, 100 kDa cut-off) at 10,000 rpm for 10 min to remove Cy3 that remained unreacted. The antibody and the Cy3 dye bound to the antibody were quantitated using intrinsic extinction coefficients at 280 nm and 552 nm, respectively (e.g., 150,000 M−1 cm−1 for Cy3). That is, the absorbance of Cy3-α-EPO, and the quotient of the absorbance divided by the extinction coefficient were used to determine the concentration of the antibody and the number of the Cy3 dye bound to one molecule of the antibody.

### Example 1-2

### Preparation of Fluorescently Labeled Biomaterial

The MAA lectin was labeled with the fluorescent dye Cy5-NHS ester to afford Cy5-MAA. The same procedure as in Example 1-1 was repeated, with the exception of using different protein and dye. The lectin and the Cy5 dye bound to the lectin were quantitated using intrinsic extinction coefficients at 280 nm and 650 nm (e.g., 250,000 M−1 cm−1 for Cy5).

### Example 1-3

### Preparation of Glycoprotein-Captured Support

As a glycoprotein comprising various carbohydrate level, EPO isoforms with various sialic acid contents were used. In this regard, EPO with the highest sialic acid content was treated with sialidase (Clostridium perfringens dialidase-agarose (Sigma)) to give EPO isoforms with various sialic acid contents. In an EP tube, 250 μL of an EPO protein (2 mg/mL) with a pI of 3.5-4.2 was mixed with 500 μL of 50 mM sodium phosphate buffer and 100 μL of sialidase-agarose. The mixture was incubated for 0, 2, 5 and 10 min with agitating gently. After centrifugation at 12,000 rpm for 3 min to remove the sialidase-agarose, the separated sialic acid was removed by centrifuging at 10,000 rpm for 10 min 3-4 times against a microfilter (Microcon YM-30, 30 kDa cut-off). The number of sialic acid on the EPO was reduced with the extension of the time of enzyme treatment. EPO was named EPO isoform-1 after enzyme treatment for 0 min, EPO isoform-2 after enzyme treatment for 2 min, EPO isoform-3 after enzyme treatment for 5 min, and EPO isoform-4 after enzyme treatment for 10 min. EPO isoform-1 almost remained intact whereas EPO isoform-4 was almost void of sialic acid.

These EPO isoforms with various sialic acid contents were diluted from 100 μg/mL to 3.1 μg/mL by half and spotted in a 3×3 matrix pattern onto ultrathin nitrocellulose support. Following incubation at 37° C. for 1 hr, the support was blocked with 1% BSA in PBS at room temperature for 1 hr, washed three times with Tris-Buffered Saline Tween-20 (TBST) and distilled water, and slowly dried in a nigrogen gas atmosphere.

### Example 1-4

### Analysis of Level of Glycoprotein and Content of Specific Carbohydrate on Support

The Cy3-α-EPO prepared in Example 1-1 was diluted to a concentration of 10 μg/mL in a solution of 0.5% Tween 20 in sodium phosphate (pH 7.0), and the support was incubated with the dilution t at room temperature for 30 min and washed with TBST and distilled water. Subsequently, the Cy5-MAA prepared in Example 1-2 was diluted to a concentration of 100 μg/mL in a solution of 0.5% Tween-20 in sodium phosphate (pH 7.0) and applied to the support at room temperature for 1 hr, after which the support was washed with TBST and distilled water and slowly dried in a nitrogen gas atmosphere.

### Example 1-5

### Measurement of Fluorescent Signal on the Support

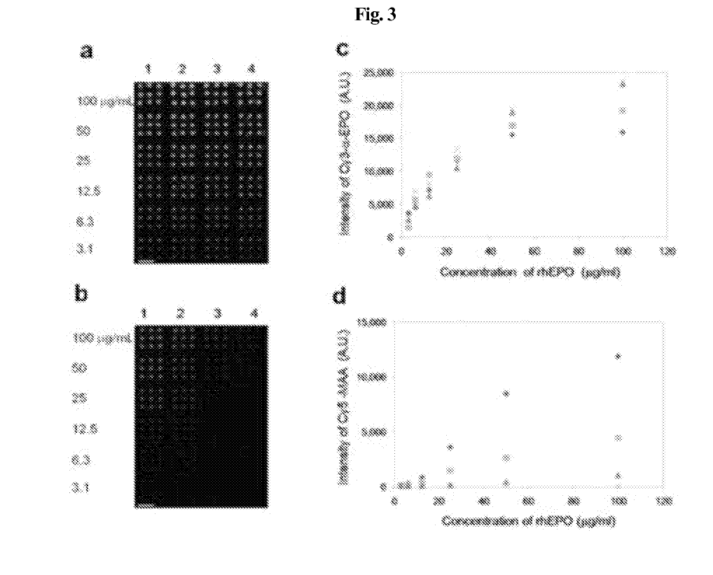

After every aspect of the above procedure, the support was inserted into GenePix 4100A Scanner (Molecular Devices) and analyzed for fluorescent signals. Scanning was performed in two wavelength conditions of 532 nm(ex)/550-600 nm(em) for Cy3-α-EPO and 635 nm(ex)/655-695 nm(em) for Cy5-MAA, and the images thus obtained were analyzed using the GenePix Pro 6.0 software (Molecular Devices) (FIG. 3). FIG. 3 shows graphs and photographs of fluorescent signal intensities as quantitatively analyzed by the glycoprotein analysis kit of the present invention. As can be seen in FIG. 3, the fluorescence intensity of Cy3-α-EPO was increased with an increase in the concentration of EPO. On the other hand, the fluorescence intensity of Cy5-MAA was found to peak in isoform-1 and to be the lowest from isoform-4 when the proteins were present at the same concentration.

These results indicate that the fluorescent signals from Cy3-α-EPO and Cy5-MAA are proportional to the level of EPO and the content of sialic acid in each spot. In addition, the intensity of Cy5 fluorescent signal detected from isoform-1 which was treated with Cy3-α-EPO and Cy5-MMA sequentially was found to be identical to that detected from isoform-1 treated with Cy5-MAA alone, indicating that the binding of Cy3-α-EPO to the glycoprotein does not interfere with the interaction of Cy5-MAA with the glycoprotein. The protein chip of the present invention allows the simultaneous exact analysis of saccharide and protein without mutual interference of respective labels.

### Example 2

### Analysis of Glycoprotein Derived from Single Cells Using Glycoprotein Analysis Kit

### Example 2-1

### Construction of Single-Cell-Based Array

The Chinese hamster ovary (CHO) cell line SCST3 which expresses EPO was plated into a PDMS microwell array, designed to have a well diameter of 30 μm, to construct a cell-based array.

Each microwell array composed of 45×45 microwells was treated with 30 μL of 50 μg/mL fibronectin (Sigma) in PBS so as to facilitate the adherence of the cells to the microwells. After the arrays were deaerated for 10 min in a vacuum chamber so as to allow the fibronectin solution to infiltrate into the wells, the fibronectin was applied to the arrays. They were washed three times with PBS and swabbed with acetone to remove fibronectin from the space between microwells. The microwells were filled with an animal cell culture medium by immersing the fibronectin-coated microwell arrays in the medium for 1 hr or longer.

After a cell culture was diluted to a density of 5×10⁵ cells/mL, 20 μL of the dilution was applied to one microwell array and incubated at 37° C. for 10 min so that cells entered microwells by gravity. Excess culture medium was removed from the microwell arrays which were then washed with a medium and placed for 6 hrs in a 37° C. (5% CO₂) incubator so as to allow the cells to adhere to the bottom of microwells.

Given a certain amount and concentration of cell to the cell culture medium, the number of animal cells entering each microwell was dependent on the diameter of microwell. In this regard, 20 μL of a cell culture medium having a density of 5×10⁵ cells/mL was applied to a microwell array in which each microwell was 35 μm in depth, and 25, 30 or 40 μm in diameter. The cells were found in approximately 65% of the microwells with one or two cells in each well of both 25 μm- and 30 μm-diameter microwell arrays. A slightly higher cell occupancy was found in 30 μm-diameter microwell arrays. Although the cells occupied a much larger number of microwells, they contained a density of two or more cells per well in a great number of microwells.

In consideration of cell number per microwell and cell occupancy, a microwell diameter of 30 μm was thereby determined as being optimal for the single cell analysis of CHO cells expressing EPO.

### Example 2-2

### Construction of a Microarray of Glycoproteins Expressed by Single Cells

For use in constructing a glycoprotein microarray, an ultrathin nitrocellulose glass substrate (PATH slide) was coated with an EPO-specific antibody. A PATH slide was incubated with 0.5 mg/mL monoclonal anti-human EPO antibody (R&D Systems) for 2 hrs at a relative humidity of 75%. A protein chip was constructed using a microengraving method. In this method, the PATH slide was immersed overnight at 4° C. in 1% BSA in TBST, and washed with distilled water just before use. The α-EPO-antibody-coated PATH slide was properly pressed against the microwall array prepared in Example 2-1 and incubated at 37° C. for 1 hr to transfer the glycoproteins secreted from single cells onto the slide. After completion of the incubation, the microwell array was detached from the slide and immersed again in the culture medium while the glass slide was washed three times with TBST and distilled water and slowly dried with nitrogen gas.

### Example 2-3

### Simultaneous Analysis of Glycoproteins Secreted from Single Cells

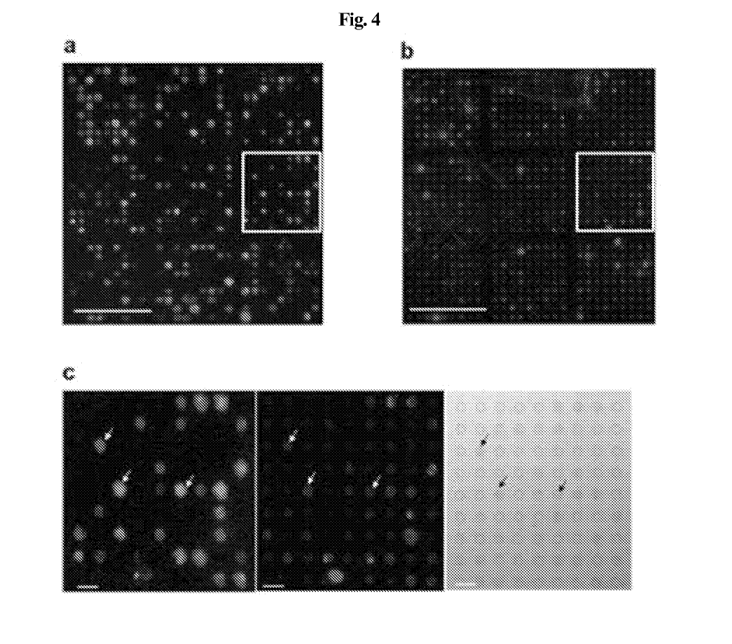

The amount of the glycoprotein secreted from single cells within the microwells constructed in Example 2-2 and the content of a specific carbohydrate in the glycoprotein were simultaneously analyzed to select single cells that expressed glycoproteins having desired glycosylation patterns. This dual probing method and the fluorescent signal measuring method were conducted in the same manner as was used with chips constructed from the EPO isoforms, with the exception that the protein chip composed of the glycoproteins secreted from single cells was used (FIG. 4). FIG. 4 shows photographs of fluorescent signals as measured by the glycoprotein analysis kit for selecting single cells in accordance with the present invention.

### Example 2-4

### Selection of Single Cells Through Statistical Analysis of Fluorescent Signals

In order to make a normal distribution for the statistical analysis of the fluorescent signals of Cy3-α-EPO and Cy5-MAA, the glycoprotein of interest secreted from single cells were analyzed on the basis of log(Cy3-α-EPO) and log(Cy5-MAA) values. Assuming that Zi and Xi are values of log(Cy5-MAA) and log(Cy3-α-EPO), respectively, at an i^{th} spot, the total amount of carbohydrate on the spot is the product of the content of carbohydrate per molecule of the glycoprotein multiplied by the concentration of the glycoprotein and thus meets the following Equation (a):

Zi=α+βxi+εi (i=1, 2, . . . , n) Equation (a)

wherein β is a ratio of log(Cy5-MAA) to log(Cy3-α-EPO), a is an intercept, and εi is an error level. On the basis of Equation (a), α′ and β′ values were calculated using the lease square estimation method, and used to deduce Equation (b) by which the number of sialic acid residues per molecule of EPO expressed by each cell can be obtained. That is, Yi, the number of sialic acid residues per molecule of EPO secreted from a single cell present in an i^{th} microwell, is simply represented by the following Equation (b):

Yi=(Zi−α′)/Xi Equation (b)

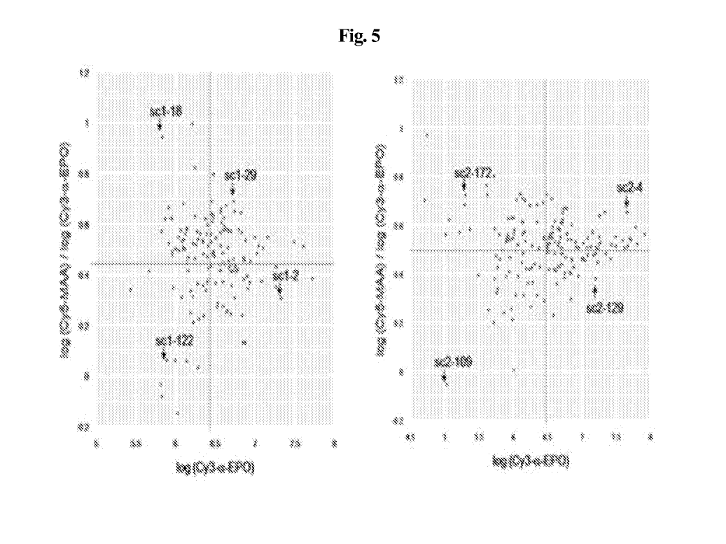

More than 1,000 microwells were examined to select 200 microwells in which single cells were placed. Yi, which is the number of sialic acid residues per EPO molecule, obtained by Equation (b), was plotted against Xi, which is the amount of EPO in each microwell, in X-Y planes (FIG. 5). FIG. 5 shows graphs of distributions of fluorescence intensities as obtained by a statistical analysis method for the selection of single cells.

The population was largely divided into four groups (subgroup-1 to 4) according to mean of X and Y values, that is, mean log(Cy3-α-EPO) and log(Cy5-MAA)/log(Cy3-α-EPO). Subgroup-1 was higher in both EPO productivity and the number of sialic acid per EPO than the mean value, subgroup-2 was lower in EPO productivity but higher in the number of sialic acid per EPO, subgroup-3 was lower in both EPO productivity and the number of sialic acid per EPO, and subgroup-4 was higher in EPO productivity, but lower in the number of sialic acid per EPO. Of the cells in each subgroup, 10 single cells showing the longest mahalanobis distance were selected.

### Example 2-5

### Isolation of Single Cells and Establishment of Cells Producing the Glycoprotein of Interest

After being heated, a capillary tube was rapidly pulled using a micropipette puller (Flamming/Brown micropipette puller (Sutter Instrument)) and elaborated at the terminal portion with heat to afford a micropipette with a diameter of 50 μm.

The microwell array was placed on an inverted microscope (DMI 3000 B, Leica) and treated with a trypsin solution at a final concentration of 10%. The tip of the micropipette was brought closely to a microwell in which the cell selected in Example 2-4 was contained so that the cell was allowed to migrate, together with the medium, into the micropipette through a capillary phenomenon. The cell was transferred to 96-well plates containing 200 μL of MEMα (Gibco) supplemented with 10% FBS in each well and cultured.

As a result, when 10 single cells were transferred, a survival rate of about 60-70% was obtained. After the cells were grown to a proper extent, they were subcultured in 24-well plates, 25-cm² T-flasks, and 75-cm² T-flasks, sequentially to a population of 10⁶ cells, and cryopreserved at −70° C. to establish a cell line producing the glycoprotein of interest.

### Example 2-6

### Biochemical Assay of the Cells

The quantity of EPO produced by the cells established in Example 2-5 was measured by ELISA. In detail, the cells were seeded at a density of 1×10⁴ cells/well to 6-well plates and grown at 37° C. for 72 hrs in an incubator during which the medium containing EPO secreted from the cells was sampled every 12 hrs and the CHO cells expressing EPO were counted. Of the anti-human EPO monoclonal antibody diluted to a concentration of 1 μg/mL in sodium carbonate (pH 9.0), 100 μL was placed in each well of 96-well plates and incubated at 37° C. for 1 hr, followed by reaction with 200 μL of 1% BSA in TBST at room temperature for 1 hr. Each of the standard solutions prepared by serially diluting 50 μg/mL EPO by ½, and the media therein, sampled every 12 hrs and containing EPO, were placed in an amount of 100 μL in each well of 96-well plates and incubated at 37° C. for 2 hrs. Each of the wells was treated at 37° C. for 30 min with 100 μL of 1 μg/mL rabbit anti-EPO IgG in TBST containing 0.3% BSA, and then again at 37° C. for 30 min with 100 μL of 0.5 μg/mL goat anti-rabbit IgG(H+L)-HRP conjugate in TBST containing 0.3% BSA. After each treatment, the well was washed three times with TBST. To each well was added 100 μL of TMB (3, 3′,5,5′-tetramethylbenzidine (Sigma)), a substrate of HRP, followed by measuring absorbance at 655 nm using a spectrophotometer (Infinite M200, Tecan). To a standard curve constructed using the absorbance values of the EPO standard solutions, the absorbance values of the samples were applied so as to determine the total amount of the glycoprotein produced. This was used to calculate a daily productivity of EPO per cell with reference to the cell count at the time of sampling.

The number of sialic acid residues per molecule of EPO produced from the newly established cell lines was analyzed using HPLC as follows. The cells were seeded at a population of 3×10⁶ cells to a 175-cm² T-flask and cultured for 3 days in a cell culture medium, and then for an additional 2 days in 20 mL of a serum-free medium (CHO-S-SFMII, Gibco) substituted for the cell culture medium. The medium was taken and centrifuged at 1,000 rpm for 10 min to remove the animal cells. The supernatant was centrifuged 3-4 times against a microfilter (Amicon Ultra, 10 kDa cut-off) at 3,000 rpm for 20 min while PBS was substituted for the medium so as to concentrate the EPO. EPO was purified from the supernatant by immunoaffinity chromatography using an anti-human EPO monoclonal antibody-bound resin, lyophilized, and stored at −20° C. The lyophilized EPO was dissolved in distilled water and treated with a weak acid to liberate sialic acid from EPO. The liberated sialic acid was selectively labeled with OPD (o-phenylenediamine-2HCl) and quantitatively analyzed using HPLC and a fluorescence detector (230 (ex), 425 (em)). Against a standard curve constructed from known fluorescence signal values of standard sialic acid solutions, the fluorescence signal values of the samples were compared so as to determine the contents of sialic acid in the samples. The number of sialic acid per EPO was calculated from the quotient of the content of sialic acid divided by the amount of EPO used. The results are summarized in Table 1, below.

### Example 3

### Cells Expressing Glycoprotein Having Desired Glycosylation Pattern

Cell lines derived from single cells exhibiting intrinsic properties were established on the basis of the selection method of Example 2-4 and the cell line establishment method of Example 2-5.

Ten single cells representative of each of the subgroups were primarily selected using the above-illustrated selection procedure and a total of four cell lines (sc1-29, sc1-18, sc1-122 and sc1-2) were analyzed by ELISA and OPD assay in the same manner as in Example 2-6 (Table 1).

TABLE 1

| Maternal | | | Productivity | Sialic acid per |
|---|---|---|---|---|
| Cell | | | (pg | EPO |
| line | Subgroup | Cell line | rhEPO/cell/day) | (nmole/nmole) |
| SCST3 | 1 | sc1-29 | 4.3 ± 0.4 | 8.7 ± 0.5 |
| | 2 | sc1-18 | 3.4 ± 0.1 | 9.0 ± 0.6 |
| | 3 | sc1-122 | 3.2 ± 0.5 | 8.2 ± 0.3 |
| | 4 | sc1-2 | 4.8 ± 0.7 | 7.8 ± 0.5 |

As can be seen in Table 1, the cell lines produced EPO at rates of 4.3±0.4, 3.4±0.1, 3.2±0.5, and 4.8±0.7 μg/cell/day, respectively. The cell lines sc1-29 and sc1-2, which were anticipated to produce EPO in high yield due to their high Cy3-α-EPO fluorescence intensities, were found to have higher productivity than did the cell lines sc1-18 and sc1-122, both being low in fluorescence intensity. In addition, the numbers of sialic acid per EPO was calculated to be 8.7±0.5, 9.0±0.6, 8.2±0.3 and 7.8±0.5 nmoles/nmole, respectively, for the cell lines, indicating that the cell lines derived from single cells with higher Cy5-MAA fluorescence intensities are richer in sialic acid. Because EPO with a higher content of sialic acid has a longer half life in vivo and thus higher pharmaceutical activity, the cell line sc1-18 expressing EPO with the highest content of sialic acid was selected and subjected to secondary selection.

The same procedure as in the primary selection was repeated to establish four cell lines (sc2-4, sc2-172, sc2-109 and sc2-129). They were also analyzed using ELISA and OPD assay in the same manner as in Example 2-6 (Table 2).

TABLE 2

| Maternal | | | Productivity | sialic acid per |
|---|---|---|---|---|
| Cell | | | (pg | EPO |
| line | Subgroup | Cell line | rhEPO/cell/day) | (nmole/nmole) |
| sc1-18 | 1 | sc2-4 | 4.0 ± 0.3 | 9.1 ± 0.6 |
| | 2 | sc2-172 | 3.4 ± 0.1 | 9.8 ± 0.1 |
| | 3 | sc2-109 | 3.2 ± 0.3 | 8.5 ± 0.1 |
| | 4 | sc2-129 | 4.4 ± 0.1 | 8.5 ± 0.1 |

As can be seen in Table 2, the cell lines the cell lines produced EPO at rates of 4.0±0.3, 3.4±0.1, 3.2±0.3 and 4.4±0.1 pg/cell/day, with sialic acid contents of 9.1±0.6, 9.8±0.1, 8.5±0.1 and 8.5±0.1 nmoles/nmole, respectively.

The highest sialic acid content, 9.8±0.1 nmoles/nmole, detected in the cell line sc2-172, was greatly increased, compared to the sialic acid content of, 8.0±0.3 and 9.0±0.6 nmoles/nmole, detected respectively in the primary and secondary maternal cell lines. That is, the repetition of the selection procedure allowed the establishment of cell lines producing EPO with higher sialic acid content.

Taken together, the data obtained in the experiments demonstrate that the glycoprotein analysis kit of the present invention, together with the selection method of the present invention, is effective for establishing a cell line producing a glycoprotein with a desired glycosylation pattern.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

1. A glycoprotein analysis kit comprising:
(A) a fluorescently labeled antibody selectively binding to a protein moiety of a glycoprotein of interest;
(B) a fluorescently labeled biomaterial selectively binding to a carbohydrate moiety of the glycoprotein of interest; and
(C) a support for immobilizing the glycoprotein of interest thereto.

2. The glycoprotein analysis kit of claim 1, wherein the biomaterial selectively binds to a specific carbohydrate structure and is selected from the group consisting of a lectin, an antibody, an aptamer and a peptide.

3. The glycoprotein analysis kit of claim 1, wherein the antibody and the biomaterial are labeled with respective fluorescent dyes which are different in absorption and emission wavelengths from each other.

4. The glycoprotein analysis kit of claim 1, wherein the glycoprotein is a purified glycoprotein or a glycoprotein isolated from single cells.

5. The glycoprotein analysis kit of claim 1, wherein the support is coated with an another antibody capable of binding to the glycoprotein of interest.

6. A method for quantitating a glycoprotein of interest, comprising:
(A) reacting a glycoprotein of interest immobilized onto a support with a fluorescently labeled antibody binding selectively to a protein moiety of the glycoprotein of interest and a fluorescently labeled biomaterial binding selectively to a carbohydrate moiety of the glycoprotein of interest; and
(B) measuring fluorescent signals generated from the antibody and the biomaterial, both being associated with the support.

7. The method of claim 6, further comprising washing the support to remove the antibody and the biomaterial which remain unreacted, between steps (A) and (B).

8. The method of claim 6, further comprising analyzing the measured fluorescent signal to determine a level of the glycoprotein and to profile glycosylation of the glycoprotein, simultaneously, after step (B).

9. A method for selecting a single cell producing a glycoprotein with a desired glycosylation pattern, comprising:
(A) culturing cells individually, said cells including the single cell displaying a glycoprotein of interest on surface thereof;
(B) bringing a support coated with an antibody specific for the glycoprotein into contact with the cells to transfer the glycoprotein secreted from the cells onto the support;
(C) reacting the glycoprotein-captured support with a fluorescently labeled antibody selectively binding to a protein moiety of the glycoprotein and a fluorescently labeled biomaterial selectively binding to a carbohydrate moiety of the glycoprotein;
(D) statistically analyzing fluorescent signals from the support; and
(E) determining the single cell having a desired glycosylation pattern, based on the analysis of the fluorescent signals, from the cells.

10. The method of claim 9, further comprising washing the support to remove the antibody and the biomaterial, both not being react with the glycoprotein, between steps (C) and (D).

11. The method of claim 9, further comprising measuring fluorescent signals from the fluorescently labeled antibody and the fluorescently labeled biomaterial, both being bound to the support, between steps (C) and (D).

12. The method of claim 9, wherein the step (D) is carried out by (i) processing measurements of fluorescent signals to set forth a normal distribution for a population distribution; and (ii) subjecting a level of the glycoprotein produced from each single cell and a number of a specific carbohydrate per molecule of the glycoprotein to relative comparison.

13. A isolated cell producing a glycoprotein having a desired glycosylation pattern, selected using the method of claim 9.
